(19) United States
(12) Patent Application Publication
(10) Pub. No.: US 2026/0076837 A1
(43) Pub. Date: Mar. 19, 2026

(54) METHOD FOR MANUFACTURING MEDICAL TAPE

(71) Applicant: YUGENKAISHA CHOURYU, Shizuoka (JP)

(72) Inventor: Toshifumi HIRASHIMA, Shizuoka (JP)

(21) Appl. No.: 18/706,009

(22) PCT Filed: Jun. 16, 2023
(86) PCT No.: PCT/JP2023/022355

§ 371 Date: Nov. 27, 2025

(30) Foreign Application Priority Data
Jun. 28, 2022 (JP) 2022-103405
Sep. 29, 2022 (JP) 2022-155771

(51) Int. Cl. A61F 13/02; A61F 13/0246

(52) U.S. Cl. CPC A61F 13/0289; A61F 13/0253; A61F 13/0266

(57) ABSTRACT

There is provided a method for manufacturing a medical tape that retains an effective shrink force specialized for each condition of a diseased area and is easy to use and wear, in order to persistently reduce a physiologic tension acting on the diseased area and its surrounding tissues, keep the state of the diseased area and its surrounding tissues in rest, and expedite natural healing. This medical tape includes an elastic base member portion having stretchability for covering a wound and a skin around the wound, an adhesion portion having a function of sticking and holding the elastic base member portion on the skin, a release portion having a function of protecting the adhesion portion, and a non-stretchable effective shrink force holding portion having flexibility and restorability, in which the effective shrink force holding portion, the elastic base member portion, the adhesion portion, and the release portion are provided in this order. The elastic base member portion internally has both a shrink force having occurred due to forming shrinkage of a solution polymer that later forms the elastic base member portion and a shrink force generated by stretching an upper base part of the elastic base member portion.

### TECHNICAL FIELD

The present invention relates to a medical tape manufacturing method coping with physiologic tension, more specifically, relates to a medical tape manufacturing method using, in a manufacturing process, a means for retaining an effective shrink force of arbitrary direction and arbitrary strength in an elastic base member portion, thereby causing the elastic base member portion to retain an effective shrink force that surpasses an internal stress accompanying forming shrinkage.

### BACKGROUND ART

A hurt formed by damaging part of a human body is called “injury”. The “injury” can be roughly classified into a “wound” which is an opened injury formed by cutting with a sharp knife or the like and “bruise” which is a closed injury formed by hitting with a blunt instrument or the like. Conventionally, in the field of the medical services, when a bottom of a wound which is an opened damage is shallow, treatment of closing and fixing the wound with an adhesive plaster, bandage, or the like is employed. When the wound bottom is deep, treatment of closing and fixing the wound with a suture thread or the like is widely employed.

In addition, the skin can be classified into three layers of the epidermis, the dermis, and the subcutaneous tissue from the upper layer. In general skin suturing, the three layers are sutured at once. Therefore, when a thick suture thread is used, a conspicuous scar is left on the skin surface in many cases. When a thin suture thread is used, wound dehiscence caused by an external force tends to increase. In order to prevent such a problem, “dermis suturing” has been selected and widely used as a suturing method. The dermis suturing is a suturing method in which the epidermis as a first layer of the skin is not sutured, but the dermis as a second layer and the subcutaneous tissue as a third layer are sutured. A suturing method in which only the epidermis as a first layer is sutured after dermis suturing is called “epidermis suturing”. In addition, in the dermis suturing, a thick suture thread (absorbent thread) that does not require thread removal is used, and a thin suture thread is selected for the epidermis suturing to minimize a scar on the skin surface.

However, even when the wound is closed and fixed through this treatment, the wound is re-opened often. Hereinafter, an event that “the wound that has been closed and fixed once is re-opened” will be referred to as “wound dehiscence”. One of the causes of the wound dehiscence is a “physiological and static sustained tension” (hereinafter, referred to as a “physiological tension”) caused by the skin and underlying muscles of the tissue surrounding the wound or the like, which acts on the tissue around the wound in the “direction of re-opening the wound”(hereinafter, referred to as a “dehiscence direction”).

Here, the wound dehiscence or the physiological tension will be described. Note that, in order to compare whether or not there is a physiological tension, “non-deteriorated tanned leather (animal hide)” is used as a sample not having the physiological tension. A non-deteriorated tanned leather having a length of 15 cm, a width of 10 cm, and a thickness of about 2 mm is prepared, and an incision having a straight line shape is made in the central part of the tanned leather with a surgical scalpel to a length of about 3 cm and a depth of about 1.5 mm. Then, the incision is sutured with a suture thread as in the case where a “skin of a living body” has an equivalent incision wound. In this case, when the suture thread is pulled with a strong force, the suture thread is broken, or the tanned leather is torn. This is also similarly applied to a case where the “skin of a living body” has an equivalent incision. When the suture thread is pulled with a strong force, the suture thread is broken, or the skin of a living body is torn. This is due to an immature stitching technique in the case of tanned leather, and an immature suturing technique in the case of skin. This problem is addressed by mastering a correct technique.

Even when the incised part of the tanned leather is stitched and closed on the basis of an appropriate stitching technique, the suture thread is broken, or the tanned leather is torn by pulling the tanned leather with a strong force perpendicularly to the close direction. Similarly, even when the incised wound of the skin is sutured with an appropriate suturing technique to close the wound, the suture thread is broken, or the skin of a living body is torn by applying a strong force to the wound in the direction to open the wound. When this happens unintentionally, the cause is an accident caused by an external force, and this is addressed by a preventive measure. This is a common phenomenon between the non-deteriorated tanned leather and the skin of a living body.

Here, the physiological tension will be visualized with reference to FIGS. 6 to 8. FIG. 6 is a schematic plan view illustrating a state of the wound surface after the skin is incised in a straight line shape. FIG. 7 illustrates the skin around the wound of FIG. 6 when it is sutured with a suture thread. FIG. 8 illustrates a force applied to a peripheral portion (wound edge) of the sutured wound of FIG. 7. Note that the black arrows in FIG. 6 refers to the physiological tension 9, the black arrows in FIG. 8 refers to a tensile force 9a including the physiologic tension, and the white arrows in FIG. 8 refers to a force 13 of attracting the wound surface by the suture thread. In addition, reference numeral 8 refers to the wound surface, reference numeral 10 refers to the suture thread, reference numeral 11 refers to the sutured wound surface, and reference numeral 12 refers to a portion through which the suture thread penetrates.

Next, the shape of the incision will be compared. In the case of tanned leather, the part incised with a surgical scalpel in a straight line shape is broken, but the shape of the incision remains linear in a straight line shape. In contrast, when the skin of a living body is incised in a straight line shape with a surgical scalpel, the wound shape is not limited to the straight line shape, and becomes an oval open shape as shown in the wound surface 8 of FIG. 6. This is due to the physiological tension 9 indicated by the black arrows in FIG. 6. In FIG. 6, for convenience, the shape of the wound surface (wound opening) 8 is an elliptically opened shape, and the direction and force of the physiologic tension 9 are illustrated in a simplified manner. The physiologic tension is a physiological and static persistent tensile force caused by tissues surrounding the wound such as a skin and underlying muscles, which is applied to the tissues surrounding the wound and acts in the direction to open the wound. Therefore, when the skin of a living body is incised in a straight line with a scalpel, the direction of the physiologic tension 9 and the magnitude of the force are variable depending on conditions such as location, direction, and depth thereof. Accordingly, the shape of the wound surface (wound opening) 8 and the direction/force of the physiologic tension 9 acting on the wound surface are not always the same, as illustrated in FIG. 6, and are not limited to the above. In addition, at the time of stitching and suturing, in the case of tanned leather having no physiological tension, the portion incised in a straight line shape may be stitched by aligning the cut ends. However, when the incised skin of a living body is sutured, it is necessary to pull the skin around the wound with a suture thread or practitioner's fingers such that the shape of the wound surface 8 (wound opening) to be sutured is changed from an oval shape to the straight line shape. This is because of the physiological tension acting in the dehiscence direction (physiological tension 9 in FIG. 6). In addition, the suture thread and the skin around the sutured wound are attractingly combined with each other after suturing by virtue of the force 13 of attracting the wound surface with the suture thread and the tensile force 9a including the physiologic tension of FIG. 8.

In addition, comparing a thread spacing or a tightening state between the stitching of tanned leather and the suturing of the skin of a living body, in the case of the suturing of skin of a living body, the coarse spacing of the sutured thread of the skin around wound produces a better prognosis than the dense spacing. Therefore, the spacing becomes inevitably coarser, and the tightness of the suture thread is loosened to prevent overtightening. For this reason, when a wound that has been successfully sutured undergoes dehiscence several days later, the patient may easily attribute the cause to the doctor's immature suturing technique, and the doctor may easily attribute it to the patient's carelessness. Therefore, they do not think that the cause is the physiological tension, and a trouble often occurs between the patient and the doctor. Accordingly, presence of the physiological tension in the suturing causes the doctor's annoyance. Note that, for wound dehiscence caused by necrosis of the skin or the like around the sutured wound due to poor circulation of blood or the like (medical tool compression wound caused by a suture thread), no effect is expected even by performing suturing again, and it is necessary to switch to conservative treatment in which all suture threads are removed, necrotic tissues are excised, the wound is covered with a covering to expect autotherapy in many cases. This is caused by the physiological tension acting on the skin around the wound, and is addressed by preventing the physiological tension acting on the skin around the wound. However, it is not easy to eliminate or reduce the physiological tension acting on the skin around the wound. Therefore, it was difficult to prevent wound dehiscence caused by the physiological tension acting in the dehiscence direction.

The above description is given to explain the wound dehiscence and the physiologic tension using a sutured wound as an example. Hereinafter, the description is turned to the physiologic tension of an un-sutured wound. Although not illustrated specifically, the un-sutured wound is affected by tissues such as a skin around the wound and underlying muscles, as well as skin division line (splitting lines formed in the process of cell division), and is subjected to physiologic tensions different in magnitude from all directions. In the case of a circular hole opened in the skin, it becomes an ellipse with a major axis in the direction in which the tension of the skin is strong because the physiologic tension is not uniform. Accordingly, even in un-sutured wounds, the same tonus as sutured wounds is always forced, and keeping the state of rest is difficult. Therefore, natural healing is delayed correspondingly, and the physiologic tension is a troublesome existence regardless of the condition of each wound sutured or not.

In some documents, a dehiscence prevention assistance tool having a function of consistently reducing the physiological tension in the dehiscence direction received by the tissue around the sutured wound and the tension acting on the suture thread associated with the physiological tension has been proposed (for example, see Patent Document 1). In this technique, a dehiscence prevention portion formed of elastic bare woven fabric (89% cotton and 11% polyurethane) is applied to the forearm by a holding portion having a function of holding the dehiscence prevention portion in close contact with the wound and the tissue around the wound by virtue of friction generated between the sutured part and the skin around the sutured part in order to reduce the physiological tension. However, in the example of the dehiscence prevention assistance tool described in Patent Document 1, a twill weave fabric using cotton threads having no elastic function is thinned and is used as a string. Therefore, when the string is too strongly tightened, it will cause a pain to the patient. When the string is too weakly tightened, it is difficult to efficiently reduce the physiological tension. In addition, there is a medical tape manufacturing method proposed for treatment of wounds (for example, refer to Patent Document 2), which is characterized by removing an internal stress holding portion to release an effective shrink force residing in an elastic base member portion. However, the above medical tape manufacturing method for treatment of wounds raises a problem that it is unable to obtain an effective shrink force that surpasses an internal stress accompanying forming shrinkage of a solution polymer. Accordingly, it is not easy to efficiently reduce the physiological tension with a conventional dehiscence prevention assistance tool, and there has been no means for appropriately addressing such a disadvantage in the current situation.

### CITATION LIST

### Patent Document

Patent Document 1: Japanese U.S. Pat. No. 4,790,091

Patent Document 2: Japanese U.S. Pat. No. 6,961,122

### SUMMARY OF INVENTION

### Technical Problems

In view of the above-described circumstances, the present invention has an object to provide a method for manufacturing a medical tape that persistently reduces a physiologic tension acting on a diseased area and its surrounding tissues, keeps the diseased area and its surrounding tissues in rest, retains an effective shrink force specialized for each condition of the diseased area for the purpose of expediting natural healing, and is easy to use and wear.

### Solution to Problem

To solve the above-described problems, the inventor made diligent studies over and over and completed the present invention as a result. That is, the manufacturing method of the present invention is a method for manufacturing a medical tape that includes an elastic base member portion having stretchability for covering a wound and a skin around the wound, an adhesion portion having a function of sticking and holding the elastic base member portion on the skin, a release portion having a function of protecting the adhesion portion, and a non-stretchable effective shrink force holding portion having flexibility and restorability. The effective shrink force holding portion, the elastic base member portion, the adhesion portion, and the release portion are provided in this order. The method includes the steps of: layering a solution polymer that later forms the elastic base member portion over a substantially arcuate concave inner surface of the effective shrink force holding portion bent into substantially arcuate concave shape in cross section under an external force, thereby obtaining a two-layered structure configured by the effective shrink force holding portion and the solution polymer, drying the solution polymer to complete film formation of the elastic base member portion and lamination of the effective shrink force holding portion and the elastic base member portion, thereby obtaining a two-layered structure configured by the effective shrink force holding portion bent into substantially arcuate concave shape in cross section and the elastic base member portion, and subsequently, removing the external force applied on the effective shrink force holding portion to cause the effective shrink force holding portion to exhibit restorability, thereby obtaining a two-layered structure configured by the elastic base member portion in a state of retaining the effective shrink force and the effective shrink force holding portion, and the effective shrink force retained in the elastic base member portion is released by removing the effective shrink force holding portion at a time of use. In the manufacturing method of the present invention, any process other than the above-described processes may be arbitrary and is not particularly limited as long as the effects of the present invention can be exerted. Note that the effective shrink force refers to a shrink force that causes the elastic base member portion to shrink in a direction resisting the physiologic tension, and is effective to reduce the physiologic tension.

Further, the medical tape of the present invention is a medical tape including an elastic base member portion having stretchability for covering a wound and a skin around the wound, an adhesion portion having a function of sticking and holding the elastic base member portion on the skin, a release portion having a function of protecting the adhesion portion, and a non-stretchable effective shrink force holding portion having flexibility and restorability, in which the effective shrink force holding portion, the elastic base member portion, the adhesion portion, and the release portion are provided in this order. This medical tape can be obtained by the manufacturing method of the present invention.

### Advantageous Effects of Invention

The medical tape obtained by the manufacturing method of the present invention retains and utilizes the effective shrink force that surpasses the internal stress accompanying forming shrinkage of the solution polymer in the elastic base member portion. To realize this, it is characterized in that the manufacturing processes include layering the solution polymer that later forms the elastic base member portion over the substantially arcuate concave inner surface of the effective shrink force holding portion bent into substantially arcuate concave shape in cross section under external force, thereby obtaining the two-layered structure configured by the effective shrink force holding portion and the solution polymer, drying the solution polymer to complete film formation of the elastic base member portion and lamination of the effective shrink force holding portion and the elastic base member portion, thereby obtaining the two-layered structure configured by the effective shrink force holding portion bent into substantially arcuate concave shape in cross section and the elastic base member portion, subsequently, removing the external force applied on the effective shrink force holding portion to cause the effective shrink force holding portion to exhibit restorability to stretch the elastic base member portion, and cause both the shrink force occurring due to forming shrinkage of the solution polymer and the shrink force generated by stretching the elastic base member portion to serve as the effective shrink force of the medical tape, thereby obtaining the two-layered structure configured by the elastic base member portion in the state of retaining the effective shrink force and the effective shrink force holding portion, and retaining the effective shrink force that surpasses the internal stress accompanying forming shrinkage of the solution polymer in the elastic base member portion, by the function of the effective shrink force holding portion. Incorporating the means for persistently reducing the physiologic tension into the medical tape as its function, in particular, configuring the tape so as to cover a diseased area and a skin around the diseased area, can efficiently reduce the physiologic tension when the tape is sticked while covering the diseased area and the skin around the diseased area as a surface. Therefore, it is possible to efficiently obtain the medical tape capable of persistently reducing the physiologic tension.

In addition, since the above-described medical tape has the function of retaining the effective shrink force in the elastic base member portion in the process of manufacturing the medical tape, it is possible at the time of use to stick the medical tape on the skin while retaining the effective shrink force in the elastic base member portion. In addition, by removing the effective shrink force holding portion after the adhesion to the skin with the adhesion portion, the effective shrink force retained in the elastic base member portion can be released to the diseased area and the skin around the diseased area, subjecting to the adhesion with the adhesion portion. This released effective shrink force not only efficiently acts in the direction resisting the physiologic tension to cause the elastic base member portion to shrink in a direction opposite to the direction to open the wound, but also causes the diseased area and the skin around the diseased area to shrink in the direction opposite to the direction to open the wound. Therefore, the skin around the diseased area can be loosened, with respect to the diseased area, from the direction opposite to the direction to open the wound. The physiologic tension acting on the skin around the diseased area can be persistently reduced. Further, this medical tape has the function of reducing the physiologic tension on the entire tape surface, and can adhere to and cover the diseased area and the skin around the diseased area as a surface. Therefore, the function of the released effective shrink force extends not only to the diseased area and the skin around the diseased area covered by the medical tape but also to a skin around the adhesion location of the medical tape, and generates a force of attracting the skin around the wound by the effective shrink force. This makes it possible to keep the diseased area and its surrounding tissues at rest, and therefore natural healing can be expedited. In addition, physiological properties of the skin include extending when a persistent extension load is applied and shrinking when the persistent extension load is removed. Therefore, removing the physiologic tension serving as the persistent extension load acting on the wound and the skin around the wound to loosen the skin around the wound causes the skin to shrink depending on its physiological properties. Accordingly, the usage of the medical tape is not limited to treatment of wounds, and this tape is usable to improve the cicatrix shrinkage (scar) or the like caused by post-healing granulation tissue.

In addition, the medical tape obtained by the manufacturing method of the present invention, that is, the medical tape of the present invention, can retain the effective shrink force specialized for each condition of the diseased area. Therefore, for example, in the case of a sutured wound, the direction of a tensile force including a relatively strong physiologic tension, acting on a diseased area and a skin around the diseased area, generally coincides with the direction of suture threads. Therefore, in the case of deeply incising a skin in a straight line and then suturing a wound in a direction perpendicular to the direction of incision while pulling the wound opened in an oval shape with fingers and suture threads or the like, a tensile force including a physiologic tension of perpendicular direction (one direction) being the direction of the suture threads becomes a dominant tensile force. Accordingly, for straight-line shaped sutured wounds, a medical tape retaining a unidirectional effective shrink force and specialized for these straight-line shaped sutured wounds is selected and used. Further, in the case of diametrically suturing a wound with an opening of perfect circle shape and a deep wound floor while pulling wound edges with fingers and suture threads or the like, a tensile force including a physiologic tension of diametrical direction (omni-direction) being the direction of suture threads becomes a relatively strong tensile force. Therefore, for diametrically sutured wounds, a medical tape retaining an omnidirectional effective shrink force and specialized for these diametrically sutured wounds is selected and used. With the selection and usage described above, the released effective shrink force efficiently acts in the direction resisting each tensile force including the physiologic tension and causes the elastic base member portion to shrink in a direction opposite to the direction in which the wound may be opened. In addition, by causing the wound and the skin around the wound to shrink in the direction opposite to the direction to open the wound, the skin around the sutured wound can be loosened in the direction opposite to the direction to open the wound, with respect to the sutured wound. As a result, the tensile force including the physiologic tension acting on the skin around the sutured wound and the tensile force acting on suture threads accompanying the above tensile force can be persistently reduced.

Further, the condition of a diseased area may include a wound being not sutured. In the case of an un-sutured wound having a straight-line shape, a medical tape retaining a unidirectional effective shrink force and specialized for straight-line shaped wounds, in which the direction of the effective shrink force is perpendicular to the direction of the wound, is selected and used. In the case of an un-sutured wound having a perfect circle shape, a medical tape retaining an omnidirectional effective shrink force and specialized for perfect circle shaped un-sutured wounds, in which the direction of the effective shrink force is diametrical to the direction of the wound, is selected and used. As a result, the released effective shrink force efficiently acts in the direction resisting each physiologic tension, and causes the elastic base member portion to shrink in the direction resisting the physiologic tension. In addition, causing the wound and the skin around the wound to shrink in the direction resisting the physiologic tension can loosen the skin around the wound, with respect to the wound, from the direction resisting the physiologic tension. As a result, the physiologic tension acting on the skin around the wound can be persistently reduced. Further, persistently reducing the physiologic tension makes it possible to eliminate the need for suturing depending on the condition of each wound. It is also expected that it copes with contused wounds, bite wounds, bedsores, and the like, which are generally difficult to suture. According to the manufacturing method of the present invention, it is possible to efficiently manufacture medical tapes.

### BRIEF DESCRIPTION OF DRAWINGS

FIG. 1 is a schematic cross-sectional diagram illustrating an effective shrink force holding portion 5, which is used for a medical tape according to a first embodiment of the present invention.

FIG. 2 is a schematic cross-sectional diagram illustrating a manufacturing process of the medical tape according to the first embodiment of the present invention, in which the process includes applying a solution polymer 1 that later forms an elastic base member portion 2 to a substantially arcuate concave inner surface of the effective shrink force holding portion 5 bent into substantially arcuate concave shape in cross section under an external force, thereby obtaining a two-layered structure configured by the effective shrink force holding portion 5 and the solution polymer 1.

FIG. 3 is a schematic cross-sectional diagram illustrating a manufacturing process of the medical tape according to the first embodiment of the present invention, in which the process includes completing film formation of the elastic base member portion 2 and lamination of the effective shrink force holding portion 5 and the elastic base member portion 2, thereby obtaining a two-layered structure configured by the effective shrink force holding portion 5 bent into substantially arcuate concave shape in cross section and the elastic base member portion 2.

FIG. 4 is a schematic cross-sectional diagram manufacturing process of the medical tape according to the first embodiment of the present invention, which illustrates processes of removing the external force applied on the effective shrink force holding portion 5 to cause the effective shrink force holding portion 5 to exhibit restorability, thereby obtaining a two-layered structure configured by the elastic base member portion 2 in a state of retaining the effective shrink force and the effective shrink force holding portion 5, and illustrates a state in which a fundamental configuration of the medical tape having a two-layered structure is completed.

FIG. 5 is a schematic cross-sectional diagram illustrating the medical tape according to the first embodiment obtained by the manufacturing method of the present invention, which illustrates a completed state of a four-layered medical tape according to the first embodiment, obtained from the fundamental configuration of the medical tape having the two-layered structure illustrated in FIG. 4, by the manufacturing method of the present invention including laminating an adhesion portion 3 on an upper layer of the elastic base member portion 2 and subsequently laminating a release portion 4.

FIG. 6 is a schematic plan view illustrating a state of a wound surface after incision of a skin in a straight line.

FIG. 7 is a schematic plan view illustrating a state of the skin around the wound illustrated in FIG. 6, which is sutured with suture threads.

FIG. 8 is a schematic plan view illustrating forces applied to a peripheral portion (wound edges) of the sutured wound illustrated in FIG. 7.

FIG. 9 is a schematic plan view illustrating the medical tape according to the first embodiment, obtained by the manufacturing method of the present invention.

FIG. 10 is a schematic plan view illustrating the medical tape according to the first embodiment illustrated in FIG. 9, obtained by the manufacturing method of the present invention and applied to the sutured wound illustrated in FIG. 7.

FIG. 11 is a schematic cross-sectional diagram illustrating an effect of using the medical tape according to the first embodiment, obtained by the manufacturing method of the present invention.

### DESCRIPTION OF EMBODIMENTS

In continuation of diligent studies, the inventor has reached the following findings. In the case of a sutured wound, in order to prevent dehiscence of the sutured wound, it is important to persistently reduce a physiologic tension in the direction to open the wound, which acts on a skin around the sutured wound or suture threads. In addition, it is also necessary to keep the skin around the sutured wound at rest in order to expedite natural healing after suturing. However, keeping the state of rest is difficult because the skin around the sutured wound is persistently subjected to the physiologic tension. Here, loosening the skin around the sutured wound, with respect to the sutured wound, from the direction opposite to the direction to open the wound, to persistently reduce the tensile force including the physiologic tension acting on the skin around the sutured wound and the tensile force acting on suture threads accompanying the above tensile force, serves an effective means for preventing the wound dehiscence caused by suture thread cutting subsequent to the suturing and the wound dehiscence caused by necrosis or the like of the skin around the sutured wound by the suture threads.

Further, there are wounds in various conditions, as described below. For example, contused wounds and bite wounds are generally difficult to suture. In the case of suturing such a wound, there will be multiple directions in which the wound may be opened. In other words, the direction to open the wound cannot be identified. Further, in a state where a wound is not sutured, the physiologic tension may act in all directions. Moreover, bedsores and the like are wounds that are forced to be not sutured. Therefore, it is important to efficiently reduce the physiologic tension acting on a skin around the wound. To cope with the wounds in such various conditions, first, the inventor considered that it was essentially necessary to obtain the elastic base member portion in a state of retaining the effective shrink force in an arbitrary direction, in which the shrink force that surpasses an internal stress accompanying forming shrinkage of a solution polymer is the effective shrink force of the medical tape. Therefore, the inventor focused on the properties of the solution polymer used for a medical tape and serving as a raw material for the elastic base member portion later having stretchability and the properties of the effective shrink force holding portion having flexibility and restorability corresponding to change of shape. With a combination of these, the inventor considered a means for retaining the effective shrink force that surpasses the internal stress accompanying forming shrinkage of the solution polymer in the elastic base member portion. For example, when the solution polymer is film-formed without contacting with a non-stretchable material, the solution polymer freely shrinks in all directions and no internal stress accompanying forming shrinkage occurs in the film-formed elastic base member portion. On the other hand, when the solution polymer is applied to an upper layer of a non-stretchable material and is film-formed, and concurrently lamination of the elastic base member portion and the non-stretchable material is completed, the solution polymer cannot freely shrink at a part (lower base part of the solution polymer) where the non-stretchable material and the solution polymer are in contact with each other. Therefore, the internal stress accompanying forming shrinkage occurs in the film-formed elastic base member portion, at the part (lower base part of the solution polymer) where the non-stretchable material and the solution polymer are in contact with each other.

Therefore, bending the effective shrink force holding portion having a function of retaining the effective shrink force in the elastic base member portion and made of the material having flexibility and restorability corresponding to change of shape into a substantially arcuate concave shape in cross section under an external force, applying a solution polymer that later forms the elastic base member portion to the substantially arcuate concave inner surface, and completing film formation of the elastic base member portion and lamination of the effective shrink force holding portion and the elastic base member portion can generate an internal stress accompanying forming shrinkage due to properties of the solution polymer serving as the raw material for the elastic base member portion, at a part (lower base part of the solution polymer) where the effective shrink force holding portion and the solution polymer are in contact with each other. The internal stress accompanying forming shrinkage generated in the above-described process is retained, by the function of the effective shrink force holding portion, as part of the effective shrink force, in the lower base part of the elastic base member portion. Subsequently removing the external force applied on the effective shrink force holding portion enables the effective shrink force holding portion to exhibit restorability and causes the two-layered structure configured by the effective shrink force holding portion bent into substantially arcuate concave shape in cross section under the external force and the elastic base member portion to return to the shape before the external force is applied (a horizontal shape according to the first embodiment of the present invention). This makes it possible to stretch the upper base part of the film-formed elastic base member portion, which is not in contact with the effective shrink force holding portion, and generate a shrink force corresponding to the amount of stretching in the upper base part of the elastic base member portion. Accordingly, both the shrink force due to the internal stress accompanying forming shrinkage generated in the lower base part of the elastic base member portion and the shrink force obtained by the restorability of the effective shrink force holding portion in the upper base part of the elastic base member portion can be utilized as the effective shrink force of the medical tape, and can be retained as the effective shrink force that surpasses the internal stress accompanying forming shrinkage of the solution polymer in the elastic base member portion. In addition, when the shape of the effective shrink force holding portion is bent into the substantially arcuate concave shape under the external force, the elastic base member portion can retain the effective shrink force whose direction is unidirectional or omni-directional depending on the applied external force, the shape of a substantially arcuate concave workbench, and the like. Further, changing the shape of the effective shrink force holding portion, the shape of the substantially arcuate concave workbench, the amount of the solution polymer applied to the effective shrink force holding portion, or the like enables the elastic base member portion to retain an arbitrary effective shrink force. The above-described findings derived the means for retaining, in the elastic base member portion, the effective shrink force that surpasses the internal stress accompanying forming shrinkage of the solution polymer as well as the effective shrink force of arbitrary direction and arbitrary strength.

Here, loosening the skin around the diseased area, with respect to the diseased area, from the direction resisting the physiologic tension, to persistently reduce the physiologic tension acting on the diseased area and the skin around the diseased area serves as an effective means for preventing the wound dehiscence by suture thread cutting subsequent to the suturing, in the case where the condition of the diseased area is a sutured wound, and the wound dehiscence caused by necrosis or the like of the skin around the sutured wound by suture threads, and for relieving tonus of the wound from the physiologic tension that always forces tonus in the case where the condition of the diseased area is an un-sutured wound. Incorporating this effective means as a function into a medical tape configured to adhere to and cover a diseased area and a skin around the diseased area as a surface can provide the entire tape surface with the function of reducing the physiologic tension. Therefore, it is possible to efficiently reduce the physiologic tension, and persistently reduce the physiologic tension acting on the diseased area and the skin around the diseased area. Regardless of the condition of the diseased area sutured or not, the diseased area and its surrounding tissues can be kept at rest, and therefore natural healing can be expedited. Further, physiological properties of the skin include extending when a persistent extension load is applied and shrinking when the persistent extension load is removed. Therefore, removing the physiologic tension serving as the persistent extension load acting on a wound and a skin around the wound to loosen the skin around the wound causes the skin to shrink depending on its physiological properties. Accordingly, the usage of the medical tape is not limited to treatment of wounds, and the tape is usable to improve the cicatrix shrinkage (scar) or the like caused by post-healing granulation tissue.

This makes it possible, in the case where the condition of the diseased area is a sutured wound, to persistently reduce the tensile force including the physiologic tension acting on the skin around the sutured wound and the tensile force acting on suture threads accompanying this tensile force, and prevent the wound dehiscence by suture thread cutting subsequent to the suturing and the wound dehiscence caused by necrosis or the like of the skin around the sutured wound by suture threads. Further, in the case where the condition of the diseased area is an un-sutured wound, it is possible to relieve the tonus of the wound from the state of being always forced to the tonus, by physiologic tensions different in magnitude from all directions. Further, persistently reducing the physiologic tension makes it possible to eliminate the need for suturing depending on the condition of the wound, and it is also expected that it copes with contused wounds, bite wounds, bedsores, and the like, which are generally difficult to suture. It is also possible to improve scars and the like after healing. From this point of view, it is recognized that, in the conventional wound dehiscence prevention assistance tool and in the manufacturing method, the physiologic tension acting on the diseased area has not been considered.

Therefore, in order to provide a medical tape incorporating a function of persistently reducing the physiologic tension acting on a diseased area and a skin around the diseased area in a manufacturing process, the inventor selected a solution polymer material, as a material for this medical tape, which covers the wound and the skin around the wound, can be used for medical purposes, as a raw material for the elastic base member portion having stretchability, has stretchability after film formation, and further resists the physiologic tension acting on the skin around the sutured wound in the wound dehiscence direction, and can obtain an effective shrink force caused by the internal stress accompanying forming shrinkage. Next, the inventor selected a non-stretchable material, as a material for the effective shrink force holding portion, from materials having flexibility and restorability corresponding to change of shape, so that it is possible, in a manufacturing process, to apply a solution polymer that later forms the elastic base member portion and complete film formation and lamination of the elastic base member portion, and exert a function of retaining the effective shrink force in the elastic base member portion. Next, the inventor selected a medical-use adhesive agent having a function of sticking and holding the elastic base member portion on the skin, as a material for an adhesion portion, and further selected a release sheet having a function of protecting the adhesion portion, as a material for a release portion, and then conceived to use the functions of respective materials to configure a medical tape manufacturing method.

By combining these materials and, in medical tape manufacturing processes, through processes of layering a solution polymer that later forms the elastic base member portion over a substantially arcuate concave inner surface of the effective shrink force holding portion bent into substantially arcuate concave shape in cross section under an external force, thereby obtaining a two-layered structure configured by the effective shrink force holding portion and the solution polymer, and drying the solution polymer to complete film formation of the elastic base member portion and lamination of the effective shrink force holding portion and the elastic base member portion, thereby obtaining a two-layered structure configured by the effective shrink force holding portion bent into substantially arcuate concave shape in cross section and the elastic base member portion, and through a subsequent process of removing the external force applied on the effective shrink force holding portion to cause the effective shrink force holding portion to exhibit restorability and return the shape of the effective shrink force holding portion to the shape before forming, thereby obtaining a two-layered structure retaining the effective shrink force of arbitrary direction and arbitrary strength in the elastic base member portion and configured by the elastic base member portion in the state of retaining the effective shrink force and the effective shrink force holding portion, and by the function of the effective shrink force holding portion, a fundamental configuration of a medical tape having a two-layered structure retaining the effective shrink force that surpasses the internal stress accompanying forming shrinkage of the solution polymer in the elastic base member portion is completed. Next, through a process of laminating the adhesion portion and the release portion over an upper layer of the elastic base member portion having the two-layered structure, a medical tape having a four-layered structure according to the first embodiment of the present invention is completed.

The medical tape obtained by the manufacturing method of the present invention has the effective shrink force holding portion, the elastic base member portion, the adhesion portion, and the release portion provided in this order. At the time of using the medical tape of the present invention, the release portion is removed, and in a state where the elastic base member portion covers a diseased area and a skin around the diseased area, the adhesion portion adheres to the skin. Next, the effective shrink force holding portion is removed. The effective shrink force holding portion plays a role of retaining the effective shrink force in the elastic base member portion. Therefore, removing the effective shrink force holding portion enables the effective shrink force retained in the elastic base member portion to be released to the diseased area and the skin around the diseased area, on which the adhesion portion is sticked. At this time, the released effective shrink force efficiently acts in the direction resisting the physiologic tension to cause the elastic base member portion to shrink in the direction resisting the physiologic tension and cause the diseased area and the skin around the diseased area to shrink in the direction resisting the physiologic tension, and further loosen the skin around the diseased area, with respect to the diseased area, from the direction resisting the physiologic tension. As a result, the physiologic tension acting on the diseased area and the skin around the diseased area can be persistently reduced. Accordingly, it is possible to incorporate the means for persistently reducing the physiologic tension acting on the diseased area and the skin around the diseased area into the medical tape as its function in the manufacturing process. Providing the entire tape surface with the function of reducing the physiologic tension can efficiently reduce the physiologic tension when the tape adheres to and covers the diseased area and the skin around the diseased area as a surface. Further, based on the finding that the effective shrink force serves for loosening the skin around the diseased area, with respect to the diseased area, from the direction resisting the physiologic tension, and therefore the physiologic tension acting on the diseased area and the skin around the diseased area can be persistently reduced, the present invention has been completed. The term “loosening the skin” as used here does not necessarily mean a visually recognizable level, and also includes an insignificant level recognizable by using a magnifying lens or microscope. In addition, the term “tape” as used here refers to a long and thin band-shaped tape having a narrow width, and this shape is not limited to the above and variable depending on the purpose of use.

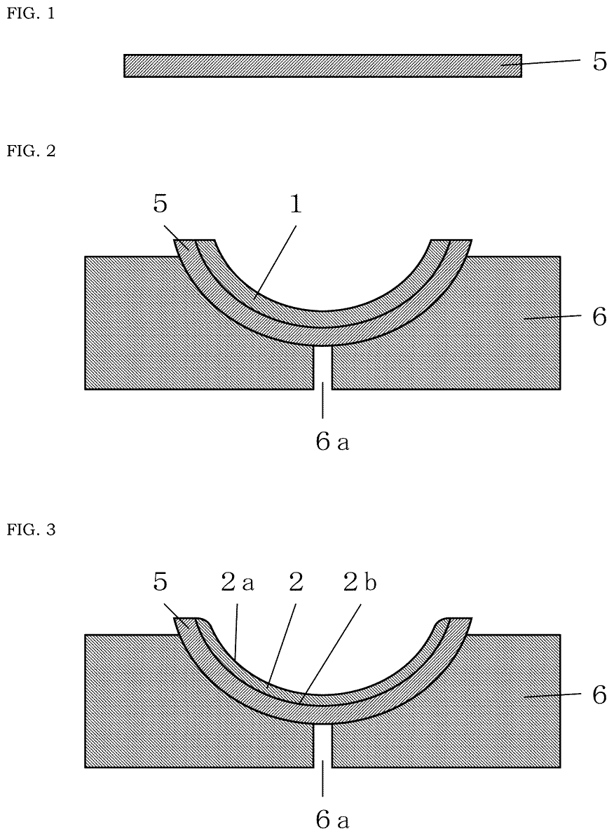

The manufacturing method of the present invention includes bending, with a substantially arcuate concave workbench 6, the effective shrink force holding portion 5 formed in a horizontal shape (FIG. 1) into a substantially arcuate concave shape in cross section under an external force, applying the solution polymer 1 that later forms the elastic base member portion 2 to the substantially arcuate concave inner surface of the effective shrink force holding portion 5, thereby obtaining a two-layered structure configured by the effective shrink force holding portion 5 and the solution polymer 1 (FIG. 2), and drying the solution polymer 1 to cure it and complete film formation of the solution polymer 1, thereby obtaining the film formation completed solution polymer as the elastic base member portion 2, and further completing lamination of the elastic base member portion 2 and the effective shrink force holding portion 5, thereby obtaining a two-layered structure configured by the effective shrink force holding portion 5 bent into substantially arcuate concave shape in cross section and the elastic base member portion 2. At this time, as the curing of the solution polymer 1 progresses, not only the elastic base member portion 2 extending in the film thickness direction but also an upper base part 2a (surface not in contact with the effective shrink force holding portion 5) of the elastic base member portion gradually shrink due to forming shrinkage of the solution polymer 1. The shape of the elastic base member portion 2 at the time when the film formation of the elastic base member portion 2 and the lamination to the effective shrink force holding portion 5 have been completed reveals that, as illustrated in FIG. 3, the film thickness of the elastic base member portion 2 is thinner than the thickness of the solution polymer 1, and the upper base part 2a of the elastic base member portion is shorter than the upper base part of the solution polymer 1. In addition, the lower base part 2b of the elastic base member portion cannot freely shrink because it is in contact with the effective shrink force holding portion 5. Therefore, the internal stress accompanying forming shrinkage of the solution polymer 1 is retained, as part of the effective shrink force, in the lower base part 2b of the elastic base member portion, by the function of the effective shrink force holding portion 5 (FIG. 3).

The term “substantially arcuate concave” as used here means that the cross-sectional shape of the effective shrink force holding portion being bent under external force is a concave arc shape, and this shape is not particularly limited as long as the effects of the present invention can be exerted.

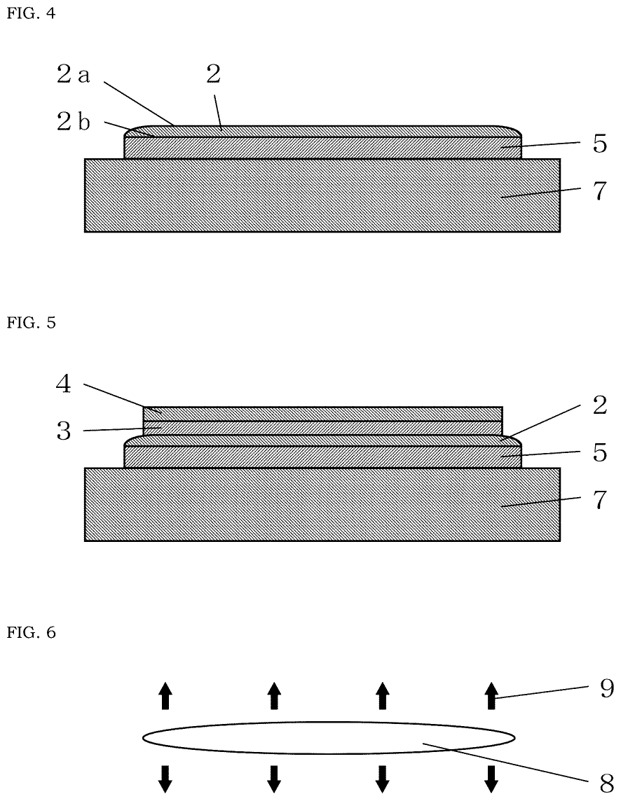

Next, the two-layered structure configured by the effective shrink force holding portion 5 bent into substantially arcuate concave shape in cross section and the elastic base member portion 2 is moved from the substantially arcuate concave workbench 6 to a horizontal workbench 7, to remove the external force applied on the effective shrink force holding portion 5 so as to cause the effective shrink force holding portion 5 to exhibit restorability (restoration force), thereby obtaining a two-layered structure with a cross-sectional shape being a horizontal shape and configured by the effective shrink force holding portion 5 and the elastic base member portion 2 (FIG. 4). At this time, it is possible to stretch the upper base part 2a (surface not in contact with the effective shrink force holding portion 5) of the elastic base member portion by the restorability (restoration force) of the effective shrink force holding portion 5. The shrink force generated by stretching the upper base part 2a of the elastic base member portion can be retained, as part of the effective shrink force, in the upper base part 2a of the elastic base member portion. The cross-sectional shape of the elastic base member portion 2 is a trapezoid slightly thinner than the elastic base member portion 2 illustrated in FIG. 3. This enables the elastic base member portion 2 to retain, as the effective shrink force, both the shrink force occurring due to forming shrinkage of the solution polymer 1 and the shrink force generated by stretching the upper base part 2a of the elastic base member portion. As a result, the elastic base member portion 2 can retain the effective shrink force that surpasses the internal stress accompanying forming shrinkage of the solution polymer. With the termination of the above process, the fundamental configuration of the medical tape having the two-layered structure is completed (FIG. 4). The physiologic tension acting in the diseased area and the skin around the diseased area does not necessarily mean a visually recognizable level, and also includes an insignificant level recognizable by using a magnifying lens or microscope level. Therefore, the effective shrink force retained in the elastic base member portion 2 may be insignificant. The effects of the present invention can be sufficiently exerted even when it is insignificant.

Hereinafter, examples of the present invention will be described in detail with reference to attached drawings, although the present invention is not limited to these. In appropriate selection/combination of materials configuring a medical tape and manufacturing processes, the present invention includes selecting, as an effective shrink force holding portion, a non-stretchable plate-shaped plastic film having flexibility and restorability corresponding to change of shape and having a function of retaining the effective shrink force in an elastic base member portion (FIG. 1). The present invention includes subsequently applying, with a substantially arcuate concave workbench, a solution polymer (urethane resin liquid and crosslinking agent liquid) that is a raw material for a polyurethane film that later forms the elastic base member portion to a substantially arcuate concave inner surface of the effective shrink force holding portion bent into substantially arcuate concave shape in cross section under an external force, thereby obtaining a two-layered structure configured by the effective shrink force holding portion 5 and the solution polymer 1 (FIG. 2). The present invention includes subsequently drying the solution polymer to cure it and complete film formation of the solution polymer, thereby obtaining the film formation completed solution polymer as the elastic base member portion, and further completing lamination of the elastic base member portion and the effective shrink force holding portion, so that an internal stress accompanying forming shrinkage occurs, due to properties of the solution polymer serving as the raw material for the elastic base member portion, at a part where the effective shrink force holding portion and the solution polymer are in contact with each other (lower base part of the solution polymer), and the generated internal stress accompanying forming shrinkage is retained, by the function of the effective shrink force holding portion, as part of the effective shrink force, in the elastic base member portion (FIG. 3).

Next, the external force applied on the effective shrink force holding portion is removed to cause the effective shrink force holding portion to exhibit restorability, thereby stretching the upper base part (surface not in contact with the effective shrink force holding portion) of the elastic base member portion. Both the shrink force occurring due to forming shrinkage of the solution polymer and the shrink force generated by stretching the upper base part of the elastic base member portion are retained, as the effective shrink force, by the function of the effective shrink force holding portion. As its function, at the time of using the medical tape, the effective shrink force holding portion is removed to release the effective shrink force retained in the elastic base member portion, which surpasses the internal stress accompanying forming shrinkage of the solution polymer. As a result, a fundamental configuration of the medical tape having a two-layered structure constituted by the effective shrink force holding portion and the elastic base member portion is completed (FIG. 4). Further, the present invention includes laminating an adhesion portion and a release portion successively on an upper layer of the elastic base member portion having the two-layered structure illustrated in FIG. 4, thereby completing a medical tape having a four-layered structure and retaining the effective shrink force according to the first embodiment of the present invention (FIG. 5). At the time of use, the medical tape covers a wound and a skin around the wound with the adhesion portion, and after adhesion to the skin, the effective shrink force holding portion is removed. The effective shrink force holding portion enables the effective shrink force retained in the elastic base member portion to be released in the direction resisting the physiologic tension, thereby reducing the physiologic tension acting in the wound dehiscence direction. The above is the basis of the present invention. The present invention essentially requires retaining the effective shrink force in the elastic base member portion in the medical tape manufacturing process.

The medical tape according to the first embodiment of the present invention illustrated in FIGS. 1 to 5 is configured by the solution polymer (urethane resin liquid and crosslinking agent liquid) 1 serving as the raw material for the elastic base member portion 2, the elastic base member portion 2 film-formed of the solution polymer (urethane resin liquid and crosslinking agent liquid) 1 serving as the raw material for the elastic base member portion 2, having stretchability, and covering a wound and a skin around the wound, the adhesion portion 3 having the function of sticking and holding the elastic base member portion 2 on the skin, the release portion 4 having the function of protecting the adhesion portion 3, and the non-stretchable effective shrink force holding portion 5 having flexibility and restorability corresponding to change of shape and having the function of retaining the effective shrink force in the elastic base member portion 2, in which the effective shrink force holding portion 5, the elastic base member portion 2, the adhesion portion 3, and the release portion 4 are provided in this order from the lower layer.

FIG. 1 is a schematic cross-sectional diagram illustrating the effective shrink force holding portion 5, which is used for the medical tape according to the first embodiment of the present invention. The medical tape according to the first embodiment uses, as the effective shrink force holding portion 5, a non-stretchable plastic film having flexibility and restorability corresponding to change of shape and having the function of retaining the effective shrink force in the elastic base member portion 2, and formed in a horizontal shape. Reference numeral 5 denotes the effective shrink force holding portion.

FIG. 2 is a schematic cross-sectional diagram illustrating a manufacturing process of the medical tape according to the first embodiment of the present invention, which illustrates a state of applying the solution polymer 1 that later forms the elastic base member portion 2 to the substantially arcuate concave inner surface of the effective shrink force holding portion 5 bent into substantially arcuate concave shape in cross section under external force, thereby obtaining the two-layered structure configured by the effective shrink force holding portion 5 and the solution polymer 1. At this time, the cross-sectional shape of the solution polymer (urethane resin liquid and crosslinking agent liquid) 1 applied to the substantially arcuate concave inner surface of the effective shrink force holding portion 5 is a substantially arcuate concave that is similar to that of the effective shrink force holding portion 5 positioned at the lower layer, as illustrated in FIG. 2. Reference numeral 1 denotes the solution polymer. Reference numeral 5 denotes the effective shrink force holding portion. Reference numeral 6 denotes the substantially arcuate concave workbench. Reference numeral 6a denotes the substantially arcuate concave workbench exhaust port.

FIG. 3 is a schematic cross-sectional diagram illustrating a manufacturing process of the medical tape according to the first embodiment of the present invention, which illustrate a state of completing film formation of the elastic base member portion 2 and lamination of the effective shrink force holding portion 5 and the elastic base member portion 2, thereby obtaining the two-layered structure configured by the effective shrink force holding portion 5 bent into substantially arcuate concave shape in cross section and the elastic base member portion 2. The shape of the elastic base member portion 2 at the time when the film formation of the elastic base member portion 2 and the lamination to the effective shrink force holding portion 5 have been completed reveals that, as illustrated in FIG. 3, the film thickness of the elastic base member portion 2 is thinner than the thickness of the solution polymer 1, and the upper base part 2a of the elastic base member portion is shorter than the upper base part of the solution polymer 1. In addition, the lower base part 2b of the elastic base member portion cannot freely shrink because it is in contact with the effective shrink force holding portion 5. Therefore, the internal stress accompanying forming shrinkage of the solution polymer 1 is retained, as part of the effective shrink force, in the lower base part 2b of the elastic base member portion, by the function of the effective shrink force holding portion 5. Reference numeral 2 denotes the elastic base member portion. Reference numeral 2a denotes the upper base part of the elastic base member portion. Reference numeral 2b denotes the lower base part of the elastic base member portion. Reference numeral 5 denotes the effective shrink force holding portion. Reference numeral 6 denotes the substantially arcuate concave workbench. Reference numeral 6a denotes the substantially arcuate concave workbench exhaust port.

FIG. 4 is a schematic cross-sectional diagram illustrating a manufacturing process of the medical tape according to the first embodiment of the present invention, which illustrates a state in which the fundamental configuration of the medical tape having the two-layered structure of the elastic base member portion 2 in the state of retaining the effective shrink force and the effective shrink force holding portion 5 is completed. The manufacturing process of FIG. 4 includes moving the two-layered structure configured by the elastic base member portion 2 and the effective shrink force holding portion 5 from the substantially arcuate concave workbench 6 illustrated in FIG. 3 to the horizontal workbench 7, thereby removing the external force applied on the effective shrink force holding portion 5 to cause the effective shrink force holding portion 5 to exhibit restorability, and returning the shape of the effective shrink force holding portion 5 to the horizontal shape that is the shape before application of the external force. At this time, a shrink force occurs in the upper base part 2a (surface not in contact with the effective shrink force holding portion 5) of the elastic base member portion, due to stretching of the upper base part 2a of the elastic base member portion. Accordingly, both the shrink force occurring due to forming shrinkage of the solution polymer 1 and the shrink force generated by stretching the upper base part 2a of the elastic base member portion can be retained, as the effective shrink force, in the elastic base member portion 2, and the effective shrink force that surpasses the internal stress accompanying forming shrinkage of the solution polymer can be retained in the elastic base member portion 2. Further, the cross-sectional shape of the elastic base member portion 2 becomes the trapezoid slightly thinner than the elastic base member portion 2 illustrated in FIG. 3, and as a result, the fundamental configuration of the medical tape having the two-layered structure has completed. Reference numeral 2 denotes the elastic base member portion. Reference numeral 2a denotes the upper base part of the elastic base member portion. Reference numeral 2b denotes the lower base part of the elastic base member portion. Reference numeral 5 denotes the effective shrink force holding portion. Reference numeral 7 denotes the horizontal workbench.

FIG. 5 is a schematic cross-sectional diagram illustrating a completed state of the medical tape according to the first embodiment of the present invention, which illustrates the state of the four-layered medical tape according to the first embodiment, obtained from the fundamental configuration of the medical tape having the two-layered structure illustrated in FIG. 4, completed by the manufacturing method of the present invention including laminating the adhesion portion 3 on the upper layer of the elastic base member portion 2 and subsequently laminating the release portion 4. Reference numeral 2 denotes the elastic base member portion. Reference numeral 3 denotes the adhesion portion. Reference numeral 4 denotes the release portion. Reference numeral 5 denotes the effective shrink force holding portion. Reference numeral 7 denotes the horizontal workbench.

Through the manufacturing processes illustrated in FIGS. 3 and 4, the function of the effective shrink force holding portion has enabled the medical tape to retain both the shrink force generated by the internal stress accompanying forming shrinkage of the solution polymer occurring in the lower base part of the elastic base member portion and the shrink force generated by the restorability of the effective shrink force holding portion in the upper base part of the elastic base member portion, as the effective shrink force, in the elastic base member portion, thereby obtaining the elastic base member portion that is in the state of retaining the effective shrink force. The procedure of using the medical tape includes: (1) removing the release portion; (2) sticking the adhesion portion on a wound and a skin around the wound; and (3) removing the effective shrink force holding portion. When the effective shrink force holding portion is removed according to this procedure for use, the effective shrink force retained in the elastic base member portion is released to the wound and the skin around the wound, on which the adhesion portion is sticked. The released effective shrink force acts in the direction resisting the physiologic tension acting in the wound dehiscence direction, causes the elastic base member portion to shrink in the direction resisting the physiologic tension, causes the wound and the skin around the wound to shrink in the direction resisting the physiologic tension applied in the wound dehiscence direction, loosens the skin around the sutured wound, with respect to the sutured wound, from the direction opposite to the wound dehiscence direction, and can persistently reduce the physiologic tension acting on the skin around the sutured wound in the wound dehiscence direction as well as the tensile force acting on suture threads accompanying this physiologic tension. In addition, the medical tape has the function of reducing the physiologic tension on the entire tape surface, and can adhere to and cover the skin as a surface. Therefore, the released effective shrink force acts not only on the wound and the skin around the wound covered by the medical tape but also on a skin around the adhesion location of the medical tape, and accordingly serves as a force of attracting the skin around the wound by the effective shrink force. This makes it possible to provide a method for manufacturing a medical tape, which can keep a wound and its surrounding tissues at rest, reduce the physiologic tension acting on the wound and the skin around the wound applied in the wound dehiscence direction, enhance the effect of preventing the wound dehiscence accompanying the physiologic tension, and is easy to use and wear, regardless of the condition of each wound sutured or not.

Specifically, according to the first embodiment of the present invention, the urethane resin liquid and the crosslinking agent liquid are selected as the solution polymer 1, the polyurethane film film-formed of the solution polymer 1 to cover the wound and the skin around the wound and having stretchability is selected as the elastic base member portion 2, an acrylic adhesive agent having the function of sticking and holding the elastic base member portion 2 on the skin is selected as the adhesion portion 3, a release sheet having a non-stretching function and obtained by coating, with a release agent, the surface of a high-quality paper having the function of protecting the adhesion portion 3 is selected as the release portion 4, and a rectangular non-stretchable plastic film formed in a horizontal shape having flexibility and restorability corresponding to change of shape and having the function of retaining the effective shrink force in the elastic base member portion 2 is selected as the effective shrink force holding portion 5. The manufacturing processes include placing a central portion of the effective shrink force holding portion 5 over the center of a substantially arcuate concave workbench exhaust port 6a, which is an exhaust port provided in the central lower portion of the substantially arcuate concave workbench 6, causing the effective shrink force holding portion 5 to hermetically contact the substantially arcuate concave workbench 6 by sucking of a discharge pump, thereby obtaining the effective shrink force holding portion 5 bent into substantially arcuate concave shape in cross section under external force, applying the solution polymer (urethane resin liquid and crosslinking agent liquid) 1 serving as the raw material for the polyurethane film that forms the elastic base member portion 2 to the substantially arcuate concave inner surface of the non-stretchable plate-shaped plastic film being the effective shrink force holding portion 5, thereby obtaining the two-layered structure configured by the effective shrink force holding portion 5 and the solution polymer 1, and then drying the solution polymer 1 to complete the film formation of the elastic base member portion 2 and the lamination of the effective shrink force holding portion 5 and the elastic base member portion 2, thereby obtaining the two-layered structure configured by the effective shrink force holding portion 5 bent into substantially arcuate concave shape in cross section and the elastic base member portion 2.

At this time, as the curing of the solution polymer 1 progresses, the shape of the solution polymer 1 changes due to forming shrinkage of the solution polymer 1, and the shape of the elastic base member portion 2 at the time when the film formation and the lamination to the effective shrink force holding portion 5 have been completed reveals that the part extending in the film thickness direction and not in contact with the effective shrink force holding portion 5 as well as the upper base part 2a (surface not in contact with the effective shrink force holding portion 5) of the elastic base member portion shrink and, as illustrated in FIG. 3, the film thickness of the elastic base member portion 2 is thinner than the thickness of the solution polymer 1, and the upper base part 2a of the elastic base member portion is shorter than the upper base part of the solution polymer 1. Further, the lower base part 2b of the elastic base member portion cannot freely shrink because it is in contact with the effective shrink force holding portion 5. The lower base part 2b of the elastic base member portion retains, as part of the effective shrink force, the internal stress accompanying forming shrinkage of the solution polymer 1, by the function of the effective shrink force holding portion 5. Next, the sucking of the effective shrink force holding portion 5 via the substantially arcuate concave workbench exhaust port 6a by the discharge pump is stopped, and the two-layered structure configured by the effective shrink force holding portion 5 and the elastic base member portion 2 is moved from the substantially arcuate concave workbench 6 to the horizontal workbench 7. Removing the external force applied on the effective shrink force holding portion 5 causes the effective shrink force holding portion 5 to exhibit restorability. The two-layered structure configured by the effective shrink force holding portion 5 bent into substantially arcuate concave shape in cross section under external force and the elastic base member portion 2 can be returned to the horizontal shape, which is the shape before the external force is applied to the effective shrink force holding portion 5. This makes it possible to stretch the upper base part 2a (surface not in contact with the effective shrink force holding portion 5) of the elastic base member portion by the restorability (restoration force) of the effective shrink force holding portion 5. The shrink force corresponding to the amount of stretching is added to the internal stress accompanying forming shrinkage of the solution polymer. By the function of the effective shrink force holding portion 5, the effective shrink force that surpasses the internal stress accompanying forming shrinkage of the solution polymer can be retained, as the effective shrink force, in the elastic base member portion 2. As a result, the fundamental configuration of the medical tape having the two-layered structure configured by the effective shrink force holding portion 5 and the elastic base member portion 2 is completed. Next, by laminating the adhesion portion 3 and the release portion 4 successively over the upper layer of the elastic base member portion 2, the medical tape having the four-layered structure according to the first embodiment of the present invention is completed.

In the first embodiment, the rectangular plate-shaped plastic film formed in the horizontal shape is used as the material for the effective shrink force holding portion, to form the effective shrink force holding portion. However, the effective shrink force holding portion may be arbitrary in material and shape depending on purpose of use, convenience, or the like. Further, in the first embodiment, by bending the effective shrink force holding portion in one horizontal direction under the external force to obtain the substantially arcuate concave shape in horizontal cross section, the direction of the effective shrink force retained in the elastic base member portion is aligned with the one horizontal direction. However, the direction of the effective shrink force retained in the elastic base member portion may be set to be omnidirectional by the following means or the like. For example, the effective shrink force holding portion is processed and molded so as to have a substantially cross shape in plane figure, to form a substantially cross-shaped effective shrink force holding portion. A workbench to be used for this substantially cross-shaped effective shrink force holding portion is hemispherical and exhibits a concave cross-sectional shape similar to that of the substantially arcuate concave workbench 6 illustrated in FIG. 2. The inside of the hemispherical workbench is recessed in such a manner that the substantially cross-shaped effective shrink force holding portion can be accurately accommodated. Further, the workbench surface is subjected to non-adhesive treatment. The concave workbench with substantially circular planer shape is formed into a substantially cross-shaped concave workbench. The substantially cross-shaped effective shrink force holding portion is bent into a concave shape in cross section under an external force, and the substantially cross-shaped effective shrink force holding portion is accurately accommodated in the internal recess of the substantially cross-shaped concave workbench. The solution polymer that later forms the elastic base member portion is applied to the concave inner surface of the substantially cross-shaped concave workbench including the substantially cross-shaped effective shrink force holding portion. The film formation of the elastic base member portion and the lamination of the effective shrink force holding portion and the elastic base member portion is completed. A two-layered structure configured by the substantially cross-shaped effective shrink force holding portion being bent into concave in cross section and the elastic base member portion is obtained. Next, this two-layered structure is moved from the substantially cross-shaped concave workbench to a horizontal workbench. The external force applied to the substantially cross-shaped effective shrink force holding portion is removed to cause the substantially cross-shaped effective shrink force holding portion to exhibit restorability, thereby obtaining a two-layered structure configured by the elastic base member portion in a state of retaining the omnidirectional effective shrink force and the substantially cross-shaped effective shrink force holding portion. The direction of the effective shrink force retained in the elastic base member portion may be an omnidirectional, and the direction of the effective shrink force provided in the elastic base member portion may be arbitrary depending on purpose of use or the like.

It is sufficient that the effective shrink force holding portion has flexibility and restorability corresponding to change of shape, retaining the effective shrink force as its function in the elastic base member portion, and can release the effective shrink force retained in the elastic base member portion by removing this effective shrink force holding portion at the time of use. It is unnecessary to cover the entire surface of the elastic base member portion. The shape is not particularly limited as long as the effects of the present invention can be exerted. In addition, depending on purpose of use, convenience, or the like, the effective shrink force holding portion may be provided with a slit or the like, and its upper layer may be provided with a guide tape or the like. Other functions may be added. The material of the effective shrink force holding portion is not particularly limited as long as it has flexibility and restorability corresponding to change of shape, retains the effective shrink force as its function in the elastic base member portion, and the effects of the present invention can be exerted.

The urethane resin liquid and the crosslinking agent liquid are used as the materials for the solution polymer in the first embodiment. Alternatively, any solution polymer material or other new materials may be used, or different solution polymer materials may be mixed or laminated, as long as the effective shrink force holding portion can retain the effective shrink force in the elastic base member portion and has stretchability after film formation, by applying it to the effective shrink force holding portion, completing the film formation of the solution polymer in the state of retaining the effective shrink force in the elastic base member portion, and concurrently completing the lamination of the elastic base member portion and the effective shrink force holding portion. Further, in the first embodiment, the amount of the solution polymer applied to the effective shrink force holding portion is substantially uniform, but the amount of the applied solution polymer may be changed between a central portion and a peripheral portion of the effective shrink force holding portion. For example, the amount of the solution polymer applied to the central portion of the effective shrink force holding portion may be increased and the amount of the solution polymer applied to the peripheral portion may be reduced. As a result, the shrink force of the effective shrink force in the central portion becomes stronger than the shrink force of the effective shrink force in the peripheral portion. Therefore, a strong effective shrink force can be efficiently exerted in the central portion of the wound. In addition, the film thickness of the elastic base member portion is thinner in the peripheral portion than in the central portion. Therefore, it is possible to prevent the medical tape from curling due to rubbing against clothing or the like during the period of adhesion.

The description of the first embodiment includes applying the solution polymer that later forms the elastic base member portion to the substantially arcuate concave inner surface of the effective shrink force holding portion bent into substantially arcuate concave shape in cross section under external force, thereby obtaining the two-layered structure configured by the effective shrink force holding portion and the solution polymer, drying the solution polymer to complete the film formation of the elastic base member portion and the lamination of the effective shrink force holding portion and the elastic base member portion, thereby obtaining the two-layered structure configured by the effective shrink force holding portion bent into substantially arcuate concave shape in cross section and the elastic base member portion. Alternatively, the manufacturing process may include applying a solution polymer that later forms an elastic base member portion to an upper layer of an effective shrink force holding portion having a horizontal shape illustrated in FIG. 1, temporarily drying (provisionally curing) the solution polymer to the extent that the solution polymer does not flow freely even if the effective shrink force holding portion is bent into a substantially arcuate concave shape in cross section under external force, thereby obtaining a two-layered structure configured by the effective shrink force holding portion and the solution polymer, subsequently bending the effective shrink force holding portion into a substantially arcuate concave shape in cross section under an external force, further drying the solution polymer to complete film formation of the elastic base member portion and lamination of the effective shrink force holding portion and the elastic base member portion, thereby obtaining a two-layered structure configured by the effective shrink force holding portion bent into substantially arcuate concave shape in cross section and the elastic base member portion. Further, the manufacturing process may include applying the solution polymer that later forms the elastic base member portion to a substantially arcuate concave inner surface of the effective shrink force holding portion while bending the effective shrink force holding portion so as to have a substantially arcuate concave shape in cross section under external force, thereby obtaining a two-layered structure configured by the effective shrink force holding portion and the solution polymer. It is sufficient if it is possible to consequently obtain the two-layered structure configured by the effective shrink force holding portion and the solution polymer, in which the solution polymer that later forms the elastic base member portion is layered over the substantially arcuate concave inner surface of the effective shrink force holding portion bent into substantially arcuate concave shape in cross section under external force.

Further, the description of the first embodiment includes sucking the effective shrink force holding portion with the discharge pump to cause the effective shrink force holding portion to hermetically contact the substantially arcuate concave workbench, thereby bending the cross-sectional shape into the substantially arcuate concave shape. However, it is sufficient that the means for bending the cross-sectional shape of the effective shrink force holding portion into the substantially arcuate concave shape is originated from the external force, and the means is not particularly limited as long as the effects of the present invention can be exerted. The term “solution polymer” as used here refers to “before the solution polymer dries, the film formation is completed, and the elastic base member portion is formed” in chronological order, and is distinguished from the elastic base member portion. Therefore, the degree of drying the solution polymer applied to the effective shrink force holding portion may be arbitrary depending on manufacturing equipment, manufacturing efficiency, and the like, and is not particularly limited as long as the effects of the present invention can be exerted. Although the acrylic adhesive agent was used as the material for the adhesion portion, it is sufficient that the adhesive agent has the function of sticking and holding the elastic base member portion on a skin and is usable for medical purposes. A treatment agent or the like may be provided in a lower layer of the elastic base member portion. The location where the adhesive agent is applied may be part or the whole depending on purpose of use, and is not restricted by the material, shape, or the like. Further, the release sheet obtained by coating the surface of a non-stretchable high-quality paper with a release agent was used as the material for the release portion. However, for example, a surface of the elastic base member portion opposite to the effective shrink force holding portion may be coated with the release agent to form the release portion, as long as it has the function of protecting the adhesion portion. A package such as wrapping paper may be used as the release portion, and there is no restriction on the material, shape, or the like.

Further, physiological properties of the skin include extending when a persistent extension load is applied and shrinking when the persistent extension load is removed. Therefore, removing the physiologic tension serving as the persistent extension load acting on a wound and a skin around the wound to loosen the skin around the wound causes the skin to shrink depending on its physiological properties. Accordingly, the usage of the medical tape is not limited to the treatment of wounds, and the tape is usable to improve the cicatrix shrinkage (scar) or the like caused by post-healing granulation tissue. Note that materials of respective portions of the medical tape, combination of the materials, functions to be added, and the like may be arbitrary depending on the purpose of use, design, convenience, or the like, without any limitation as long as the effects of the present invention can be exerted. Further, not only in wounds but also in swelling due to inflammation and the like, a swollen area and a surrounding skin are often harmed by the physiologic tension. At this time, sticking the medical tape while covering the swollen area and the surrounding skin can persistently reduce the physiologic tension acting on the swollen area and the surrounding skin. Further, the effective shrink force retained in the medical tape serves as a force for compressing the swollen area accompanying inflammation of a diseased area and the like. The function of compressing the swollen area and the function of reducing the physiologic tension have the effect of relieving pain, itching, or the like accompanying inflammation. In addition, the medical tape can have a plurality of functions such as a permeation function and a moistening function depending on purpose of use or the like. Therefore, the application of this medical tape is not limited to wounds, and the medical tape may be used in any other applications as long as the functions of the medical tape are effectively exerted. The usage of the medical tape is not limited by any application restrictions as long as the use is medically effective.

Any medical tape obtained by the manufacturing method of the present invention illustrated in FIGS. 1 to 5 has the fundamental structure of the medical tape obtained by the manufacturing method of the present invention. Adopting such a configuration can incorporate the means for persistently reducing the physiologic tension acting on a diseased area and a skin around the diseased area into the medical tape as its function. Further, it is possible to manufacture a medical tape that can retain the effective shrink force in the elastic base member portion, in a process of manufacturing the medical tape. And, at the time of use, the medical tape can be sticked on a skin in the state of retaining the effective shrink force in the elastic base member portion. Further, in this medical tape, after adhesion to the skin with the adhesion portion, by removing the effective shrink force holding portion, the effective shrink force retained in the elastic base member portion can be released to the diseased area and the skin around the diseased area, on which the adhesion portion is sticked. This released effective shrink force efficiently acts in the direction resisting the physiologic tension, and not only causes the elastic base member portion to shrink in the direction opposite to the direction to open the wound but also causes the diseased area and the skin around the diseased area to shrink in the direction opposite to the direction to open the wound. This can loosen the skin around the diseased area, with respect to the diseased area, from the direction opposite to the direction to open the wound, and can persistently reduce the physiologic tension acting on the skin around the diseased area.

Further, the medical tape has the function of reducing the physiologic tension on the entire tape surface, and can adhere to and cover the diseased area and the skin around the diseased area as a surface. Therefore, the function of the released effective shrink force extends not only to the diseased area and the skin around the diseased area covered by the medical tape but also to a skin around the adhesion location of the medical tape, and generates the force of attracting the skin around the wound by the effective shrink force. This makes it possible to provide a medical tape that can keep a diseased area and its surrounding tissues at rest, expedite natural healing, enhance the effect of preventing the wound dehiscence, and is easy to use and wear, regardless of the condition of each wound sutured or not. Note that, although the shrink force of the medical tape is expressed as “loosening the skin around the wound” or “loosening the skin around the diseased area” herein, this is not necessarily a visually recognizable level and also includes an insignificant level recognizable by using a magnifying lens or microscope. This is because the physiologic tension acting on an object wound and a skin around the wound and the tensile force acting on suture threads accompanying this physiologic tension do not necessarily mean a visually recognizable level, and also includes an insignificant level recognizable by using a magnifying lens or microscope. The same can be said for the physiologic tension acting on a diseased area and a skin around the diseased area. Therefore, the effective shrink force retained in the elastic base member portion may be insignificant. The effects of the present invention can be sufficiently exerted even when it is insignificant.

In the first embodiment, the description has been given using the exemplary medical tape having a rectangular shape. However, the shape of the medical tape may be selected freely depending on the purpose of use. For example, the medical tape may have a circular or oval shape, a roll shape, or a sheet shape and may be appropriately shaped depending on each part of a body. In addition, this medical tape basically has the configuration including, from the lower layer, the effective shrink force holding portion, the elastic base member portion, the adhesion portion, and the release portion. However, a gauze, a pad, or the like may be provided on the upper layer of the adhesion portion depending on the purpose of use, design, convenience, and the like. Although the medical tape of the present invention is basically sticked closely on the skin, the medical tape may be applied by inserting a protective material or the like for protecting the skin from a medical use adhesive agent or the like, in some cases.

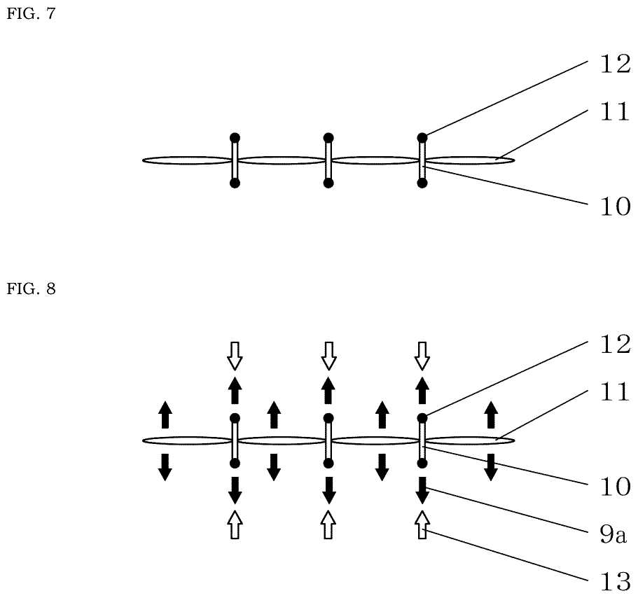

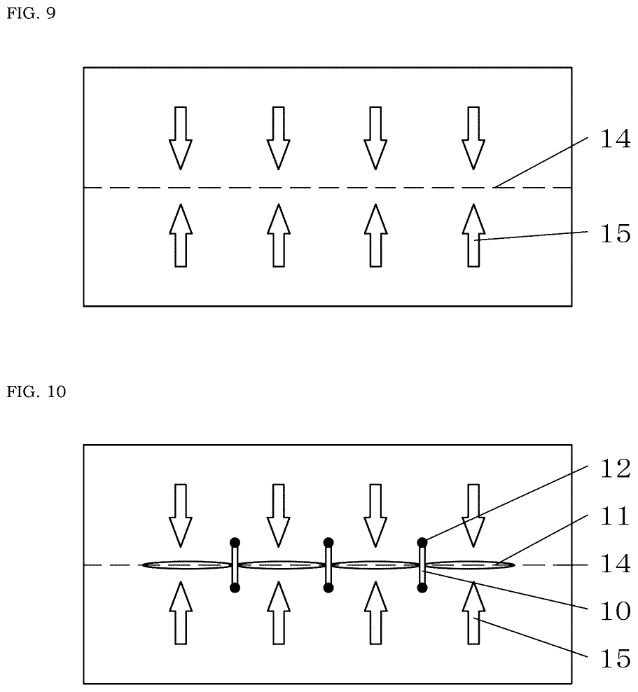

Next, the physiologic tension associated with suture threads used to suture wounds, the tensile force including this physiologic tension, the wound dehiscence, and the usage method and effects of the medical tape according to the first embodiment of the present invention will be described with reference to FIGS. 6 to 11. FIG. 6 is a schematic plan view illustrating a state of a wound surface after incision of a skin in a straight line. Reference numeral 8 denotes the wound surface. Reference numeral 9 denotes the physiologic tension, and arrows indicate directions thereof. When the skin is incised in a straight line with a scalpel, the shape of the wound surface 8 is not limited to the straight line. As illustrated in FIG. 6, the physiologic tension 9 acting on the skin around the wound makes the shape from the straight line to an oval shape. FIG. 7 is a schematic plan view illustrating a state of the skin around the wound illustrated in FIG. 6, which is sutured with suture threads. Reference numeral 10 denotes the suture thread. Reference numeral 11 denotes the sutured wound surface. Reference numeral 12 denotes the portion through which the suture thread penetrates. When suturing the wound surface 8 of FIG. 6, it is necessary to pull the wound surface 8 with suture threads, practitioner's fingers, or the like to adjust the shape thereof from the elliptically opened state to the incised straight-line shape. Since the prognosis is better when the intervals between suture threads for suturing the skin around the wound is coarse rather than dense, the spacing of suture threads becomes inevitably coarse at this time, and the state of the suture threads is moderately loosened as illustrated in FIG. 7, in order to prevent overtightening, which results in poor prognosis. Further, the physiologic tension 9 illustrated in FIG. 6 becomes a tensile force 9a including the physiologic tension as illustrated in FIG. 8, because the tensile force increases when suturing the wound surface 8 of FIG. 6 while pulling the wound surface 8 in elliptically opened state with suture threads, practitioner's fingers, or the like in order to adjust the shape thereof to the incised straight-line shape.

FIG. 8 is a schematic plan view illustrating the force applied to a peripheral portion (wound edges) of the sutured wound illustrated in FIG. 7. Reference numeral 9a denotes the tensile force including the physiologic tension, arrows indicate directions thereof, and lengths indicate magnitudes thereof. Reference numeral 10 denotes the suture thread. Reference numeral 11 denotes the sutured wound surface. Reference numeral 12 denotes the portion through which the suture thread penetrates. Reference numeral 13 denotes the force of attracting the wound surface by the suture threads, arrows indicate directions thereof, and lengths indicate magnitudes thereof. In the suturing of the wound surface 8 illustrated in FIG. 6, the skin around the wound is pulled and sutured with the suture threads, practitioner's fingers, or the like in order to adjust the shape of the wound surface 8 to be sutured from the opened state to the closed state, and the suture threads are tied. Therefore, at wound edges of a wound peripheral portion, as illustrated in FIG. 8, the force 13 of attracting the wound surface by the suture threads and the force in the direction to open the wound surface by the tensile force 9a including the physiologic tension act in a balanced state, and therefore the suture threads and the skin around the wound are always pulled each other.

Further, at the location 12 where the suture thread pierced, the force 13 of attracting the wound surface by the suture threads and the force in the direction to open the wound surface by the tensile force 9a including the physiologic tension are balanced, and therefore the wound surface is kept in a closed state. However, even on the sutured wound surface 11, the spacing between the suture threads is coarse, and at a wound surface between the suture threads where the force 13 of attracting the wound surface by the suture threads does not reach, the force in the direction to open the wound surface by the tensile force 9a including the physiologic tension is dominant, and therefore the shape of the wound surface between the suture threads becomes a small oval shape. Further, in a skin around the location 12 where the suture thread pierced, the force 13 of attracting the wound surface by suture threads and the force in the direction to open the wound surface by the tensile force 9a including the physiologic tension are balanced, and therefore the tensile force 9a including the physiologic tension is concentrated on the wound surface side of the location 12 where the suture thread pierced, that is, a skin tissue inside the suture thread. When the persistent compression by the suture thread is applied to the skin tissue on the wound surface side, this compression causes poor blood circulation or the like in the skin tissue on the wound surface side, the skin tissue falls into compression necrosis, and this compression necrosis gradually advances to the skin tissue on the wound surface side, tears the skin around the wound, causes the suture thread to be removed, and causes wound dehiscence in some cases. Under the current situation, even re-suturing is not expected to be effective for the wound dehiscence mainly caused by the necrosis of the skin around the wound or the like (medical tool compression wound caused by suture threads).

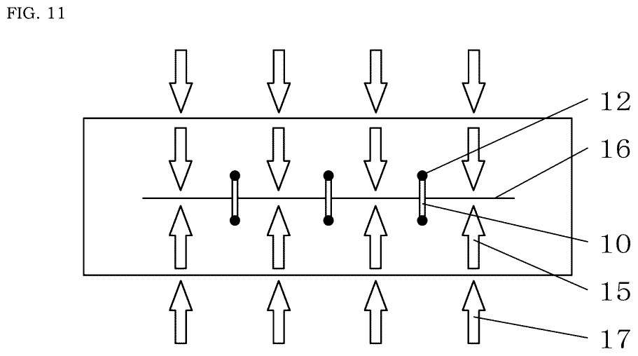

FIG. 9 is a schematic plan view illustrating the medical tape according to the first embodiment, obtained by the manufacturing method of the present invention. This medical tape is a medical tape retaining a unidirectional effective shrink force and specialized for straight-line shaped sutured wounds, because, in the case of such a straight-line shaped sutured wound, the tensile force including a physiologic tension of perpendicular direction (one direction) being the direction of suture thread becomes a dominant tensile force. Reference numeral 14 denotes the broken line provided in the center of a medical tape. Reference numeral 15 denotes the direction and magnitude of the effective shrink force. The broken line 14 provided in the center of the medical tape is printed on the effective shrink force holding portion of this medical tape, and is for facilitating the procedure of overlappingly sticking this tape on the wound at the time of use. The effective shrink force 15 is retained in the elastic base member portion, by the function of the effective shrink force holding portion. FIG. 10 is a schematic plan view illustrating the medical tape according to the first embodiment illustrated in FIG. 9, obtained by the manufacturing method of the present invention and applied to the sutured wound illustrated in FIG. 7. Reference numeral 10 denotes the suture thread. Reference numeral 11 denotes the sutured wound surface. Reference numeral 12 denotes the portion through which the suture thread penetrates. Reference numeral 14 denotes the broken line provided in the center of the medical tape. Reference numeral 15 denotes the direction and magnitude of the effective shrink force. First, the medical tape having a size capable of appropriately covering the sutured wound and its surrounding skin is selected. Next, the release portion is removed from the medical tape, and the sutured wound and its surrounding skin are covered by the elastic base member portion in such a manner that the broken line 14 provided in the center of the medical tape and the sutured wound surface 11 of FIG. 7 overlap each other, and the medical tape is sticked on the skin by the adhesion portion. At this time, since the effective shrink force holding portion is not removed, the effective shrink force 15 is retained in the elastic base member portion, by the function of the effective shrink force holding portion.

FIG. 11 is schematic cross-sectional diagram illustrating an effect of using the medical tape according to the first embodiment, obtained by the manufacturing method of the present invention. Reference numeral 10 denotes the suture thread. Reference numeral 12 denotes the portion through which the suture thread penetrates. Reference numeral 15 denotes the direction and magnitude of the effective shrink force. Reference numeral 16 denotes the wound surface closed by the effective shrink force. Reference numeral 17 denotes the force of attracting the skin around the wound by the effective shrink force. FIG. 11 illustrates a state where the effective shrink force 15 retained in the elastic base member portion and the force 17 for pulling the skin around the wound by the effective shrink force are exhibited, by removing the effective shrink force holding portion on which the broken line 14 provided in the center of the medical tape is printed, from the state illustrated in FIG. 10. In this medical tape, since the shrinkage direction of the effective shrink force is limited to one direction, it is possible to efficiently exhibit the unidirectional effective shrink force 15 and persistently reduce the physiologic tension acting on the skin around the sutured wound surface 11 by covering, as a surface, the skin around the sutured wound surface 11 illustrated in FIG. 10 to stick the tape to the skin, and removing the effective shrink force holding portion. Accordingly, when suture threads are used for suturing, the shape of the sutured wound surface 11 illustrated in FIG. 8, that is, the shape of the wound surface between the suture threads, is a small oval shape because the force in the direction to open the wound surface by the tensile force 9a including the physiologic tension illustrated in FIG. 8 is dominant, in the wound surface between the suture threads where the force 13 of attracting the wound surface by suture threads does not reach in FIG. 8. However, as illustrated in FIG. 11, using this medical tape can exhibit the effective shrink force 15 corresponding to the shrinkage in length in one direction from the medical tape of FIG. 10, so that the wound surface 16 closed by the effective shrink force is obtained.

In addition, the medical tape has the function of reducing the physiologic tension on the entire tape surface, and can adhere to and cover the skin as a surface. Therefore, the released effective shrink force 15 acts not only on the skin around the sutured wound surface 11 covered by the medical tape but also on the skin around the adhesion location of the medical tape, so that the force 17 for pulling the skin around the wound is generated by this effective shrink force. This makes it possible to reduce the load of the tensile force by the tensile force 9a including the physiologic tension concentratedly acting on the wound surface side of the location 12 where the suture thread pierced, that is, the skin tissue inside the suture thread. And, it is possible to avoid a virtuous circle, that is, “when the persistent compression by the suture thread is applied to the skin tissue on the wound surface side, this compression causes poor blood circulation or the like in the skin tissue on the wound surface side, the skin tissue falls into compression necrosis, and this compression necrosis gradually advances to the skin tissue on the wound surface side, tears the skin around the wound, causes the suture thread to be removed, and causes wound dehiscence in some cases”. Therefore, it becomes realistic to prevent the wound dehiscence mainly caused by necrosis of the skin around the wound (medical tool compression wound caused by suture thread). Furthermore, even for the dermis suturing described in paragraph 0003, the medical tape of the present invention becomes an effective means for minimizing scars on skin surfaces, and it becomes possible to eliminate the necessity of epidermal suturing and also avoid needle marks on skin surfaces.

### INDUSTRIAL APPLICABILITY

According to the medical tape manufacturing method of the present invention, the effective shrink force of arbitrary direction and arbitrary strength is retained in the elastic base member portion in the manufacturing process. Further, at the time of use, the medical tape adheres to and covers a diseased area and a skin around the diseased area as a surface with the adhesion portion in the state where the effective shrink force is retained in the elastic base member portion. And then, the effective shrink force holding portion is removed, thereby releasing the effective shrink force from the elastic base member portion, and persistently reducing the physiologic tension acting on the diseased area and the skin around the diseased area, so as to keep the diseased area and its surrounding tissues in rest and expedite natural healing. The usage thereof is not limited to wounds. It is possible to provide a method for manufacturing a medical tape that is easy to use and wear, and the present invention greatly contributes to the medical industry.

### REFERENCE SIGNS LIST

1 solution polymer
2 elastic base member portion
2a upper base part of elastic base member portion
2b lower base part of elastic base member portion
3 adhesion portion
4 release portion
5 effective shrink force holding portion
6 substantially arcuate concave workbench
6a substantially arcuate concave workbench exhaust port
7 horizontal workbench
8 wound surface
9 physiologic tension
9a tensile force including physiologic tension
10 suture thread
11 sutured wound surface
12 portion through which suture thread penetrates
13 force of attracting wound surface by suture thread
14 broken line in center of medical tape according to the first embodiment of the present invention
15 effective shrink force
16 wound surface closed by effective shrink force
17 force of attracting skin around the wound by effective shrink force

1. A method for manufacturing a medical tape, wherein
the medical tape includes:
an elastic base member portion having stretchability for covering a wound and a skin around the wound;
an adhesion portion having a function of sticking and holding the elastic base member portion on the skin;
a release portion having a function of protecting the adhesion portion; and
a non-stretchable effective shrink force holding portion having flexibility and restorability,
the effective shrink force holding portion, the elastic base member portion, the adhesion portion, and the release portion are provided in this order,
the method comprises the steps of:
layering a solution polymer that later forms the elastic base member portion over a substantially arcuate concave inner surface of the effective shrink force holding portion bent into substantially arcuate concave shape in cross section under an external force, thereby obtaining a two-layered structure configured by the effective shrink force holding portion and the solution polymer, and drying the solution polymer to complete film formation of the elastic base member portion and lamination of the effective shrink force holding portion and the elastic base member portion, thereby obtaining a two-layered structure configured by the effective shrink force holding portion bent into substantially arcuate concave shape in cross section and the elastic base member portion; and
subsequently, removing the external force applied on the effective shrink force holding portion to cause the effective shrink force holding portion to exhibit restorability, thereby obtaining a two-layered structure configured by the elastic base member portion in a state of retaining the effective shrink force and the effective shrink force holding portion, and
the effective shrink force retained in the elastic base member portion is released by removing the effective shrink force holding portion at a time of use.

2. A medical tape comprising:
an elastic base member portion having stretchability for covering a wound and a skin around the wound;
an adhesion portion having a function of sticking and holding the elastic base member portion on the skin;
a release portion having a function of protecting the adhesion portion; and
a non-stretchable effective shrink force holding portion having flexibility and restorability, wherein
the effective shrink force holding portion, the elastic base member portion, the adhesion portion, and the release portion are provided in this order, and
the elastic base member portion internally has a shrink force having occurred due to forming shrinkage of a solution polymer that later forms the elastic base member portion and a shrink force generated by stretching an upper base part of the elastic base member portion.
